(19) United States
(12) Patent Application Publication
(10) Pub. No.: US 2004/0064030 A1
(43) Pub. Date: Apr. 1, 2004

(54) Detection of implanted injection port

(76) Inventor: Peter Forsell, Zug (CH)

Correspondence Address: NIXON & VANDERHYE, PC, 1100 N GLEBE ROAD, 8TH FLOOR, ARLINGTON, VA, 22201-4714, US

(21) Appl. No.: 10/260,546

(22) Filed: Oct. 1, 2002

(51) Int. Cl. A61B 5/05

(52) U.S. Cl. 600/409

(57) ABSTRACT

An apparatus is disclosed for detecting an injection port subcutaneously implanted in a patient's body to enable accurate positioning of an injection needle outside the patient's body to enable the injection needle to be injected directly into the injection port. Also disclosed is a method for detecting the injection port whereby an injection needle can be positioned to be injected directly into the injection port. The apparatus includes a magnetic device that is subcutaneously implanted in the patient adjacent to the injection port to emit a local magnetic field through the patient's skin adjacent to the injection port. A magnetic detector movable externally along the patient's body is capable of detecting the local magnetic field emitted by the magnetic device. This allows the injection port to be located for the insertion of the injection needle directly into the injection port. Alternatively, the apparatus can include a magnetic detector subcutaneously implanted in the patient by the injection port and an exterior magnetic device movable along the patient's skin to emit a magnetic field that is detected by the implanted magnetic detector. Preferably, the magnetic detector includes a semiconductor circuit that is comprised of at least one Hall element. The magnetic device may be a solenoid or a permanent magnet. The injection port can be used to control a restriction device implant designed for treating reflux disease, urinary incontinence, impotence, anal incontinence or obesity.

### FIELD OF THE INVENTION

The present invention relates to an apparatus and methods for detecting an injection port subcutaneously implanted in a patient. The present invention also relates to surgical methods for treating a patient having a disease by using the apparatus.

### BACKGROUND

It is important to locate the position of an injection port connected to a hydraulically operable surgical implant in a patient to be able to accurately inject a needle of a syringe through the membrane of the injection port (or simply for the purpose of locating the exact position of the injection port, or alternatively locating the membrane of the injection port), for supplying hydraulic fluid to or withdrawing hydraulic fluid from the implant.

In the prior art, for the specific purpose of locating an injection port, a technique has been used to detect the injection port by using magnetism induced by a solenoid. More particularly, a rotating solenoid is rotated in different positions to locate the injection port. This technique has clear disadvantages such as the very large size of the apparatus used and the relatively insensitive way of locating the magnetic field.

### SUMMARY OF THE INVENTION

An object of the present invention is to provide an inexpensive apparatus and methods for accurately detecting an injection port subcutaneously implanted in a patient to enable an injection needle to penetrate the patient's skin directly into the injection port. Another object of the present invention is to provide surgical methods for treating diseases by using the apparatus.

In accordance with a first aspect of the present invention, there is provided an apparatus for detecting an injection port subcutaneously implanted in a patient comprised of a magnetic device designed to be subcutaneously implanted in the patient at the injection port for emitting a local magnetic field extending through the injection port and a portion of the patient's skin adjacent to the injection port, and a magnetic detector movable externally along the patient's body and capable of detecting the local magnetic field emitted by the magnetic device when the magnetic detector is positioned in front of the implanted injection port, the magnetic detector including a semiconductor circuit. Thus, the accurate position on the patient's skin in front of the injection port, which is hidden behind the skin, can be established using the apparatus of the present invention. With an injection needle placed in this position, it is an easy task to properly insert the injection needle through the patient's skin directly into the injection port.

Alternatively, the apparatus is comprised of a magnetic detector designed to be subcutaneously implanted in the patient at the injection port, the magnetic detector including a semiconductor circuit, an exterior magnetic device movable along the patient's skin and adapted to emit a local magnetic field through the patient's skin, the magnetic detector being capable of detecting the local magnetic field when the movable magnetic device is moved to a position in front of the implanted injection port, whereby an injection needle placed in this position is able to be inserted through the patient's skin directly into the injection port.

Thus, according to the present invention a new and easy way of detecting the position of an injection port subcutaneously implanted in a patient is provided, which enables accurate positioning of a needle or the like outside the patient's body for safe and accurate injection (contact with the membrane) in the injection port, by using magnetism and a semiconductor circuit. The injection port is arranged in connection (via a conduit) to an implant, for example a food intake restriction apparatus, implanted inside the human body. The method according to the present invention provides a very sensitive way of locating the magnetic field which is a necessity in obese people.

Alternatively, where the magnetic detector is to be implanted in the patient, a sender capable of sending information about the magnetic detector detecting the local magnetic field may be implanted in the patient.

Preferably, the magnetic detector includes a semiconductor circuit. According to a preferred embodiment of the present invention, the semiconductor circuit of the magnetic detector is comprised of at least one Hall-element. By using one or more Hall-elements, which is a special type of semiconductor known in the art, it is easy to locate the central axis of the emitted magnetic field. The magnetic detector suitably is comprised of several Hall-elements which are grouped around a central point in a triangular or square configuration. One important feature is that the Hall-elements are able to detect even a weak magnetic field emitted from the magnetic device. The described detection technique is simple, inexpensive and very accurate, and could be used for several different implants in combination with injection ports.

The magnetic device may be a solenoid or a permanent magnet, which is sending out a magnetic field. If the magnetic device is placed outside the body, the magnetic detector placed inside the body should preferably also be capable of sending information about the position of the magnetic device, directly or indirectly correlated to the intensity of magnetism to outside the body.

Conveniently, the location of the injection port, subcutaneously implanted in a patient, may be visualised on a screen, by sound or by diodes.

In accordance with a second aspect of the present invention, there is provided a method for detecting an injection port subcutaneously implanted in a patient, the method comprising the steps of: implanting a magnetic device subcutaneously in the patient at the injection port so that the magnetic device emits a local magnetic field extending through the injection port and the adjacent skin portion of the patient; and moving an exterior magnetic detector along the patient's skin to a position in which the local magnetic field emitted by the implanted magnetic device is detected by the magnetic detector. Then, an injection needle can be placed in the position where the local magnetic field has been detected to efficiently insert the needle through the patient's skin directly into the injection port.

Alternatively, the method may comprise the steps of: implanting a magnetic detector subcutaneously in the patient at the injection port; moving an exterior magnetic device along the patient's skin while it emits a local magnetic field extending through the adjacent skin portion; and using the implanted magnetic detector to detect the local magnetic field when the magnetic device is moved to a position in which the local magnetic field extends through the implanted magnetic detector and injection port.

This alternative method may further comprise implanting a sender and using the sender to send information to outside the patient's body confirming when the implanted magnetic detector detects the local magnetic field emitted by the exterior magnetic device.

In accordance with a third aspect of the present invention, there is provided a surgical method for treating a patient having a disease, comprising the steps of: insufflating the patient's abdomen with gas; implanting a hydraulically operable implant designed for treating reflux disease, urinary incontinence, impotence, anal incontinence or obesity in the abdomen by using surgical instruments through the trocars; subcutaneously implanting an injection port for supplying hydraulic fluid for the operation of the implant and a magnetic device at the injection port for emitting a local magnetic field through the injection port and the adjacent skin portion of the patient; post-operatively moving an exterior magnetic detector along the patient's body to a position in which the local magnetic field emitted by the implanted magnetic device is detected by the magnetic detector; bringing an injection needle to the position in which the local magnetic field is detected; and moving the injection needle to penetrate the patient's skin into the injection port for supplying hydraulic fluid to or withdrawing hydraulic fluid from the injection port.

Alternatively, the surgical method may comprise subcutaneously implanting a magnetic detector at the injection port and post-operatively moving an exterior magnetic device emitting a local magnetic field along the patient's body to a position in which the local magnetic field emitted by the exterior magnetic device is detected by the implanted magnetic detector.

The above described apparatuses and methods may also be designed for treating reflux disease, urinary incontinence, anal incontinence, obesity and impotence.

### BRIEF DESCRIPTION OF THE DRAWINGS

FIG. 1 shows a connection diagram for a magnetic detector according to one aspect of the present invention.

FIG. 2 schematically illustrates in a diagram the position relative to the magnet as a function of the sensor (i.e. detector) according to the present invention.

FIG. 3 illustrates an embodiment according to the present invention using several Hall-elements as the magnetic detecting device.

FIG. 4 is a schematic view of an embodiment where a magnetic device is subcutaneously implanted in the patient, and a magnetic detector movable externally along the patient's body.

FIG. 5 is a schematic view of an embodiment where a magnetic detector is subcutaneously implanted in the patient and an exterior magnetic device is movable along the patient's skin.

FIG. 6 is a schematic view of a band with a cavity defining a restriction opening for use in accordance with the invention, designed for treating reflux disease, urinary incontinence, anal incontinence or obesity.

### DETAILED DESCRIPTION OF THE INVENTION

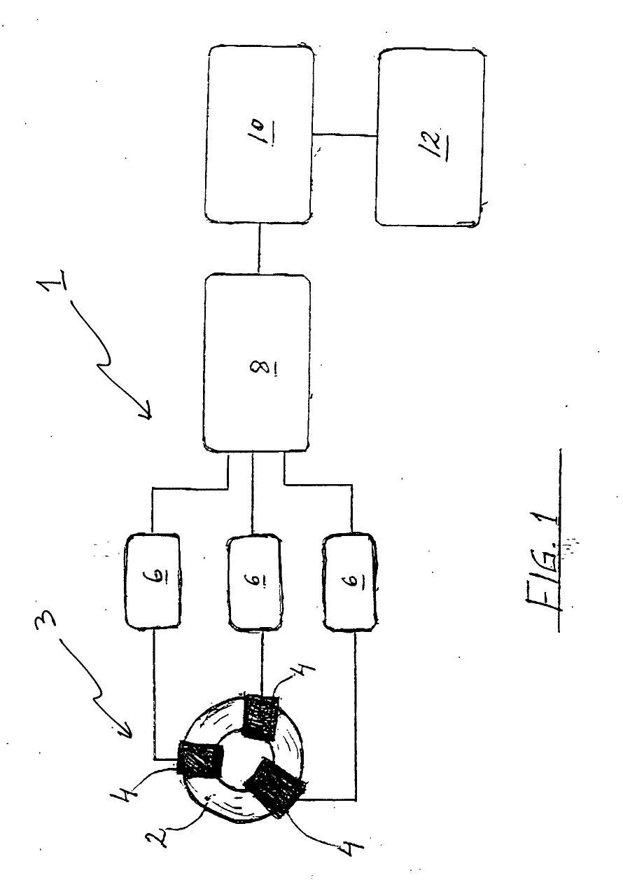

FIG. 1 shows a connection circuit 1 for a magnetic detector 3 according to the present invention. A magnetic device implanted inside a human body is a ring-magnet 2, which can be a solenoid or a permanent magnet. Located outside the body is magnetic detector 3, which is comprised of three linear magnetic field sensors 4 (such as Hall-elements or the like) grouped in a triangular configuration. Sensors 4 are connected to signal-conditioning amplifiers 6, which, in turn, is connected to an A/D-converter 8. A microprocessor 10 is then connected to A/D-converter 8. To visually display the sensor output signals of sensors 4, a display-device 12 is then connected to microprocessor 10.

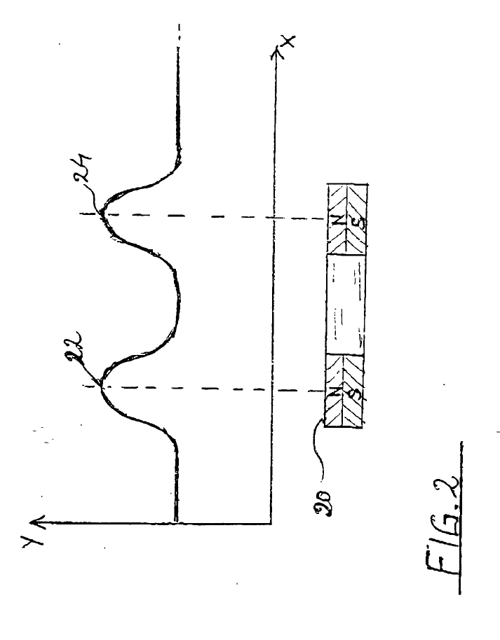

The graph shown in FIG. 2 illustrates in principle how the information obtained by detector 3 can be presented. On the X-axis in the graph is the position of detector 3 relative to magnet 2. On the Y-axis is the combined output of sensors 4. Thus, the graph of FIG. 2 shows the position “X” of detector 3 relative to magnet 2 as a function of the sensor (i.e. detector) output “Y”. To illustrate this method of sensing, a ring-magnet 20 is shown relative to the graph of FIG. 2. Ring-magnet 20 is shown in cross-section to show the positions of its magnetic northpole N and southpole S, respectively. FIG. 2 depicts the case where magnetic detector 3 (not shown in FIG. 2) has been centered in front of ring-magnet 20 and where all of the sensors 4 produce a maximum output which is shown as peaks 22, 24 in the graph of FIG. 2. Sensors 4 are connected (e.g., by a connection circuit 1 shown in FIG. 1) to display device 12, which may display the graph shown in FIG. 2, or alternatively, a numeral result from the measurements taken by sensors 4.

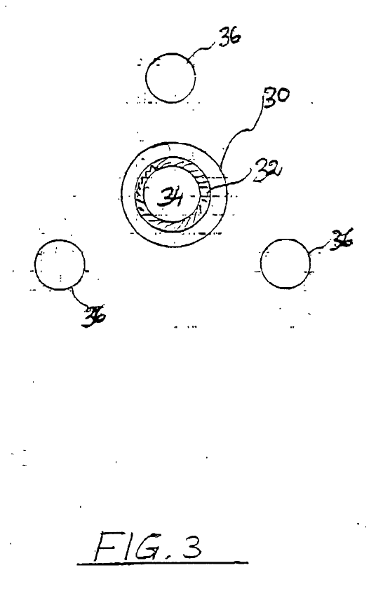

FIG. 3 shows an embodiment of the apparatus of the invention where the magnetic detector suitably comprises several Hall-elements 36 which are grouped around a central point in a triangular or square configuration. In this embodiment, the magnetic device positioned by the implanted injection port 30 is preferably a ring-shaped magnet 32 surrounding the membrane 34 of the injection port 30. The magnetic detecting device arranged outside the patient's body comprises three symmetrically arranged Hall-elements 36. When the Hall-elements 36 are placed symmetrically above and around the ring-shaped magnet 32, i.e., when the ring-shaped magnet 32 is in the center of injection port 30, information is sent, directly or indirectly correlated to the intensity of magnetism, about the location of the injection port 30.

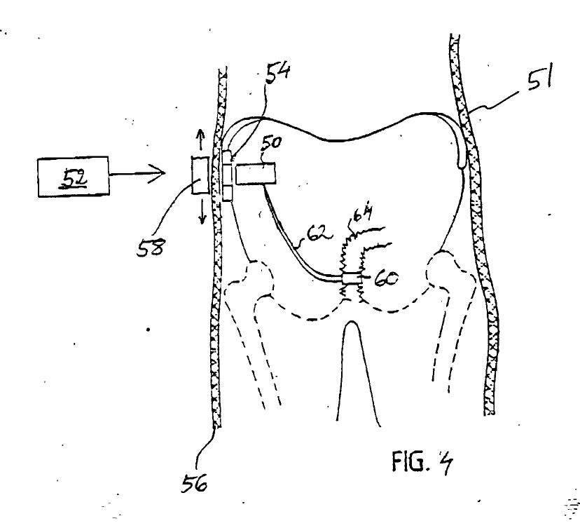

FIG. 4 shows an embodiment of the apparatus of the present invention for detecting an injection port 50 subcutaneously implanted in a patient 51 suffering from anal incontinence to enable accurate positioning of an injection needle 52 outside the patient's body for safe and accurate injection in the injection port 50. Injection port 50 is, via a conduit 62, connected to an implanted artificial sphincter 60 applied to the patient's rectum 64. The apparatus of the present invention is comprised of a magnetic device 54 subcutaneously implanted in patient 51 adjacent to injection port 50. Magnetic device 54 emits a local magnetic field extending through injection port 50 and a portion of patient 51's skin 56 adjacent to injection port 50. The apparatus of the present invention is further comprised of an external, separate magnetic detector 58 that may be manually moved along patient 51 's body to detect the local magnetic field emitted by implanted magnetic device 54. Magnetic detector 58 detects the local magnetic field when it is positioned in front of injection port 50. When this position has been determined, injection needle 52 can be located in the same position to efficiently insert the needle through patient 51's skin directly into injection port 50.

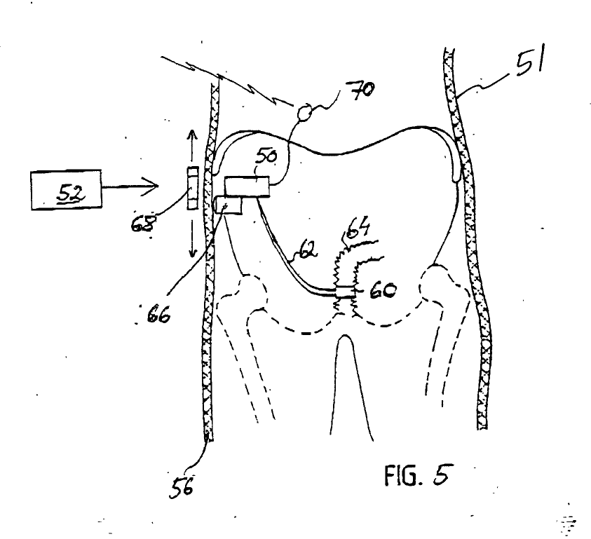

FIG. 5 shows a modification of the embodiment of FIG. 4, and is comprised of a magnetic detector 66 subcutaneously implanted in patient 51 at injection port 50. An external separate magnetic device 68 may be manually moved along patient 51 's body while emitting a local magnetic field through patient 51 's skin 56. Magnetic detector 66 is capable of detecting the local magnetic field when movable magnetic device 68 is moved to a position in front of implanted injection port 50. A sender 70 is implanted in patient 51 and sends information about the status of magnetic detector 66. Thus, when magnetic detector 66 detects the local magnetic field emitted by external magnetic device 68, sender 70 sends information confirming that magnetic device 68 is in a proper position for accurate positioning of an injection needle 52 outside the patient's body. When this position has been determined, injection needle 52 can be placed in the same position to efficiently insert it in the injection port 50.

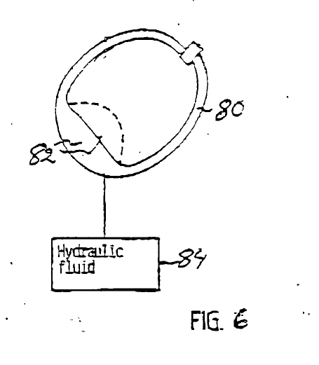

FIG. 6 shows an example of artificial sphincter 60, which is comprised of a band 80 formed into a loop around the patient's rectum (not shown in FIG. 5). Band 80 has a cavity 82 which can be inflated by supplying hydraulic fluid thereto, via implanted injection port 50, to close the rectum, and be deflated by withdrawing hydraulic fluid therefrom, to open the rectum. A hydraulic operation device 84 for operating band 80 can be powered with energy from an implanted energy receiving device. This type of band may also be used as an artificial sphincter for treating patient's suffering from heartburn and reflux disease or urinary incontinence combined with the apparatus of the invention. Furthermore, band 80 may be used for forming an adjustable constricted stoma opening in the stomach or esophagus of an obese patient or for restricting the penile exit blood flow of an impotent patient combined with the apparatus of the invention.

What is claimed is:

1. An apparatus for detecting an injection port adapted to be subcutaneously implanted in a patient, comprising a magnetic device designed to be subcutaneously implanted in the patient at the injection port for emitting a local magnetic field extending through the injection port and a portion of the patient's skin adjacent to the injection port, and a magnetic detector movable externally along the patient's body and capable of detecting said local magnetic field emitted by said magnetic device when said magnetic detector is in a position in front of the implanted injection port, said magnetic detector including a semiconductor circuit, whereby an injection needle put in said position is able to run through the patient's skin right into the injection port.

2. An apparatus according to claim 1, wherein said semiconductor circuit of said magnetic detector comprises at least one Hall-element.

3. An apparatus according to claim 2, wherein said magnetic detector comprises several Hall-elements grouped around a central point in a triangular or square configuration.

4. An apparatus according to claim 1, wherein said magnetic device is a solenoid or a permanent magnet.

5. An apparatus according to claim 1, wherein said magnetic device comprises a ring-shaped magnet.

6. An apparatus for detecting an injection port subcutaneously implanted in a patient, comprising a magnetic detector designed to be subcutaneously implanted in the patient at said injection port, said magnetic detector including a semiconductor circuit, an exterior magnetic device movable along the patient's skin and adapted to emit a local magnetic field through the patient's skin, said magnetic detector being capable of detecting said local magnetic field when said movable magnetic device is moved to a position in front of said implanted injection port, whereby an injection needle placed in said position is able to be inserted through the patient's skin directly into the injection port.

7. An apparatus according to claim 6, further comprising a sender adapted to be implanted in the patient and capable of sending information about said magnetic detector detecting said local magnetic field.

8. An apparatus according to claim 6, wherein said magnetic device is a solenoid or a permanent magnet.

9. An apparatus according to claim 6, wherein said semiconductor circuit of said magnetic detector comprises at least one Hall-element.

10. A method for detecting an injection port subcutaneously implanted in a patient, the method comprising the steps of:
implanting a magnetic device subcutaneously in the patient at the injection port so that the magnetic device emits a local magnetic field extending through the injection port and the adjacent skin portion of the patient; and
moving an exterior magnetic detector along the patient's skin to a position in which the local magnetic field emitted by the implanted magnetic device is detected by the magnetic detector.

11. A method for detecting an injection port subcutaneously implanted in a patient, the method comprising the steps of:
implanting a magnetic detector subcutaneously in the patient at the injection port;
moving an exterior magnetic device along the patient's skin while it emits a local magnetic field extending through the adjacent skin portion; and
using the implanted magnetic detector to detect the local magnetic field when the magnetic device is moved to a position in which the local magnetic field extends through the implanted magnetic detector and injection port.

12. A method according to claim 11, further comprising implanting a sender and using the sender to send information to outside the patient's body confirming when the implanted magnetic detector detects the local magnetic field emitted by the exterior magnetic device.

13. A surgical method for treating a patient having a disease, comprising the steps of:
insufflating the patient's abdomen with gas;
placing at least two laparoscopical trocars in the patient's body;
implanting a hydraulically operable implant designed for treating reflux disease, urinary incontinence, impotence, anal incontinence or obesity in the abdomen by using surgical instruments through the trocars;
subcutaneously implanting an injection port for supplying hydraulic fluid for the operation of the implant and a magnetic device at the injection port for emitting a local magnetic field through the injection port and the adjacent skin portion of the patient;
post-operatively moving an exterior magnetic detector along the patient's body to a position in which the local magnetic field emitted by the implanted magnetic device is detected by the magnetic detector;
bringing an injection needle to the position in which the local magnetic field is detected; and
moving the injection needle to penetrate the patient's skin into the injection port for supplying hydraulic fluid to or withdrawing hydraulic fluid from the injection port.

14. A surgical method for treating a patient having a disease of reflux disease, urinary incontinence, impotence, anal incontinence or obesity, comprising the steps of:
insufflating the patient's abdomen with gas;
placing at least two laparoscopical trocars in the patient's body;
implanting in the abdomen using surgical instruments through the trocars a hydraulically operable implant designed for treating the disease;
subcutaneously implanting an injection port for supplying hydraulic fluid for the operation of the implant and a magnetic detector adjacent to the injection port;
post-operatively moving an exterior magnetic device emitting a local magnetic field along the patient's body to a position in which the local magnetic field emitted by the exterior magnetic device is detected by the implanted magnetic detector;
bringing an injection needle to the position in which the local magnetic field is detected; and
moving the injection needle to penetrate the patient's skin into the injection port for supplying hydraulic fluid to or withdrawing hydraulic fluid from the injection port.
